(19) United States
(12) Patent Application Publication
(10) Pub. No.: US 2021/0007714 A1
(43) Pub. Date: Jan. 14, 2021

(54) ULTRASOUND SYSTEM FOR SHEAR WAVE IMAGING IN THREE DIMENSIONS

(71) Applicant: KONINKLIJKE PHILIPS N.V., EINDHOVEN (NL)

(72) Inventors: SHENG-WEN HUANG, OSSINING, NY (US); HUA XIE, CAMBRIDGE, MA (US); MAN NGUYEN, MELROSE, MA (US); CAROLINA AMADOR CARRASCAL, EVERETT, MA (US); JEAN-LUC JEAN-LUC FRANCOIS-MARIE ROBERT, CAMBRIDGE, MA (US); VIJAY THAKUR SHAMDASANI, KENMORE, MA (US)

(21) Appl. No.: 16/982,603

(22) PCT Filed: Mar. 4, 2019
(86) PCT No.: PCT/EP2019/055343

§ 371 Date: Sep. 21, 2020

Related U.S. Application Data
(60) Provisional application No. 62/645,909, filed on Mar. 21, 2018.

(51) Int. Cl. A61B 8/08; A61B 8/00; A61B 8/14

(52) U.S. Cl. CPC A61B 8/485; A61B 8/463; A61B 8/54; A61B 8/4488; A61B 8/469; A61B 8/14

(57) ABSTRACT

An ultrasound imaging system for analyzing tissue stiffness by shear wave measurement comprises a matrix array probe which acquires shear wave velocity data from three planes of a volumetric region of interest. The velocity data is used to color-code pixels in the planes in accordance with their estimated tissue stiffness. The planes are displayed in their relative spatial orientation in an isometric or perspective display. The positions and orientations of the planes can be changed from the system user interface, enabling a clinician to view selected planes of stiffness information which intersect the region of interest.

### TECHNICAL FIELD

This invention relates to medical ultrasound imaging systems and, in particular, to ultrasound systems which perform measurements of tissue stiffness or elasticity using shear waves.

### BACKGROUND

An advantage of ultrasound imaging and other imaging modalities is that, in addition to depicting the structure of tissue and pathology in the body, it is also possible to anatomically visualize characteristics and functionality of the tissue or pathology being imaged. This is done by acquiring two images of the anatomy, one structural and another which is parametric. The two images are then overlaid for display in anatomical registration. A basic parametric image in ultrasound is a colorflow image, whereby a B mode image of tissue structure is overlaid with a color image representing the direction and velocity of blood flow in vessels and other structure of the tissue. The structure of blood vessel walls frames the blood flow information, showing the clinician parameters of the blood flow at the locations where it is occurring. The clinician can diagnose blood flow functionality at specific locations in the body by observing parameters of the flow such as its velocity and direction at anatomical locations defined by the surrounding tissue. Other parametric imaging procedures are also well known in ultrasound, such as tissue motion imaging, contrast imaging of tissue perfusion, and strain imaging of tissue elasticity.

Another parametric imaging procedure which has evolved more recently is shear wave imaging. Like strain imaging, shear wave imaging is an elastographic technique which provides indications of tissue stiffness. For example, stiffer tissue regions of the breast or liver might be malignant or scarred, whereas softer and more compliant areas are more likely to be benign and healthy. Since the stiffness of a region is known to correlate with malignancy or benignity, and scarred or healthy cells, elastography provides the clinician with another piece of evidence to aid in diagnosis and determination of a treatment regimen.

In order to form a shear wave image, shear wave measurements are made throughout a region of interest. The physiological phenomena behind an ultrasonic shear wave measurement are as follows. When a point on the body is compressed, then released, the underlying tissue undergoes local axial displacement in the direction of the compression vector, then rebounds back when the compressive force is released. But since the tissue under the compressive force is continuously joined to surrounding tissue, the uncompressed tissue lateral of the force vector will respond to the up-and-down movement of the local axial displacement. A rippling effect in this lateral direction, referred to as a shear wave, is the response in the surrounding tissue to the downward compressive force. Furthermore, it has been determined that the force needed to push the tissue downward can be produced by radiation pressure from an ultrasound pulse, and ultrasound reception can be used to sense and measure the tissue motion induced by the shear waves. Shear wave velocity is determined by local tissue mechanical properties. The shear wave will travel at one velocity through soft tissue, and at another, higher velocity through stiffer tissue. By measuring the velocity of the shear wave at a point in the body, information is obtained as to characteristics of the tissue stiffness at that point, such as its shear elasticity modulus and Young's modulus. The laterally propagating shear wave travels slowly, usually a few meters per second or less, making the shear wave susceptible to detection, although it attenuates rapidly over a few centimeters or less. See, for example, U.S. Pat. No. 5,606,971 (Sarvazyan) and U.S. Pat. No. 5,810,731 (Sarvazyan et al.) The shear wave velocity is virtually independent of the amplitude of tissue displacement, and tissue density normally has little variance, which make the technique suitable for objective quantification of tissue characteristics with ultrasound.

### SUMMARY

Existing commercial systems which perform shear wave elastographic assessment, such as the ElastQ feature of the Epiq ultrasound system from Philips Healthcare of Andover, Mass., use planar (2D) imaging techniques. But tissues in the body are three-dimensional, not two-dimensional. Shear wave speed can change in the elevational direction as well as in the two-dimensional azimuthal and depth plane of an image. In addition, shear wave speed is direction-dependent in anisotropic tissues. Information obtained from current commercial products is thus incomplete. One way to produce a 3D shear wave velocity map is through elevational sweeping of a one-dimensional (1D) probe, and by rotating the probe by 90 degrees and performing another sweep, a 3D map of shear wave speed in the new lateral direction can be acquired as well. Such procedure is, however, slow because shear wave imaging usually runs at a low frame rate limited by thermal effects and can suffer from registration errors as well. Moreover, the tissue motion caused by shear wave travel is very subtle. Peak shear wave tissue displacements are at best about 10 μm, and under more common, less favorable circumstances are closer to 1 μm. The precision of displacement estimates for accurate shear wave measurements should be at least on the order of 100 nm. Furthermore, shear wave motion is heavily damped in tissue, which is viscoelastic in character. The tissue motion elicited by the force used for shear wave generation propagates radially in all directions perpendicular to the force vector and suffers a fall-off as a factor of 1/R in the radial directions in addition to normal attenuation caused by tissue viscosity. These factors mandate that shear wave generation and measurement be done at closely spaced intervals throughout a region of interest. When such measurements are performed throughout a volumetric region, the time required to sample a full volume is significant. And the tissue displacement resulting from shear wave travel can easily be overwhelmed by motional effects due to patient heartbeat and hand-held transducer movement.

Accordingly, it is desirable to be able to acquire and display shear wave stiffness measurements in three dimensions while retaining the acquisition frame rate and accuracy needed for a reliable diagnosis.

In accordance with the principles of the present invention, an ultrasonic shear wave imaging system is described which improves the accuracy and reliability of shear wave stiffness assessment in three dimensions. A two-dimensional (2D) matrix array transducer probe is used to acquire shear wave velocity data in three planes of a region of interest. Pixels in the planes are color-coded in accordance with their measured tissue stiffness and displayed in their spatial orientations in an isometric or perspective display. The positions and orientations of the planes can be changed by the system user interface, enabling a clinician to view selected planes of stiffness information which intersect in the region of interest.

In the drawings:

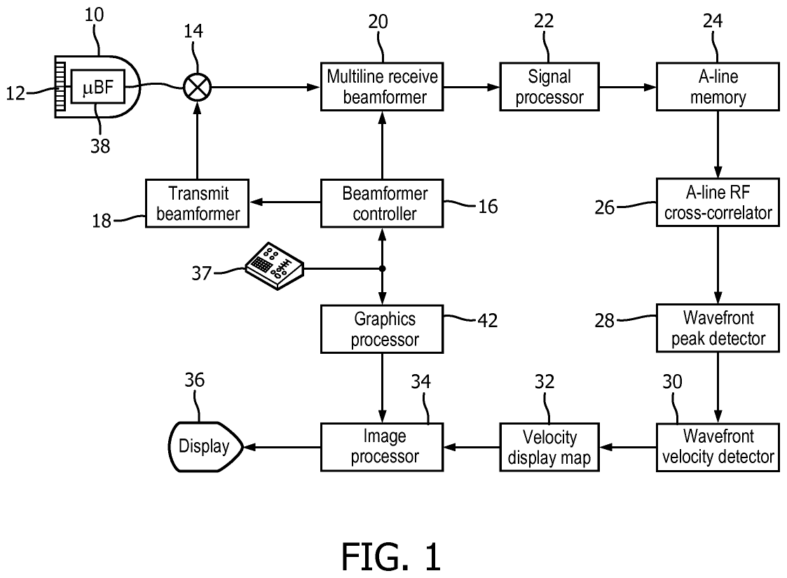

FIG. 1 illustrates in block diagram form an ultrasound imaging system constructed to perform shear wave measurement in three dimensions in accordance with the principles of the present invention.

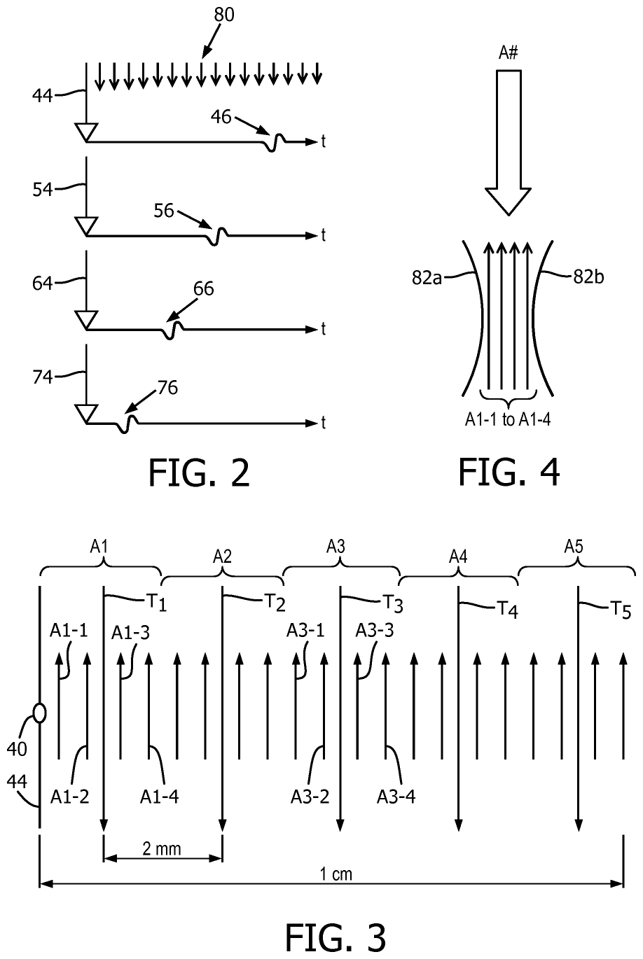

FIG. 2 illustrates a sequence of pulse pulses along a push pulse vector, the resultant shear wavefront, and a series of tracking pulse vectors.

FIG. 3 illustrates four laterally adjacent groups of 4× multiline tracking pulse vectors.

FIG. 4 illustrates the transmission and reception of a 4× multiline pulse for the production of four adjacent multiline tracking pulse vectors in a region of interest.

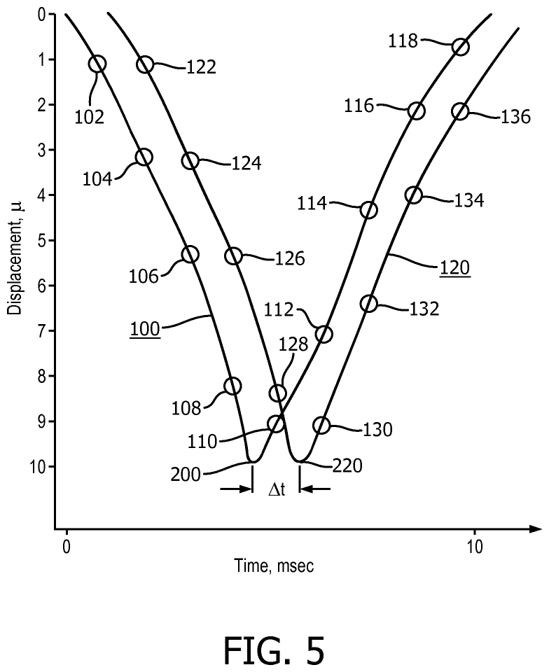

FIG. 5 illustrates a shear wave displacement curve at two locations as it progresses through tissue.

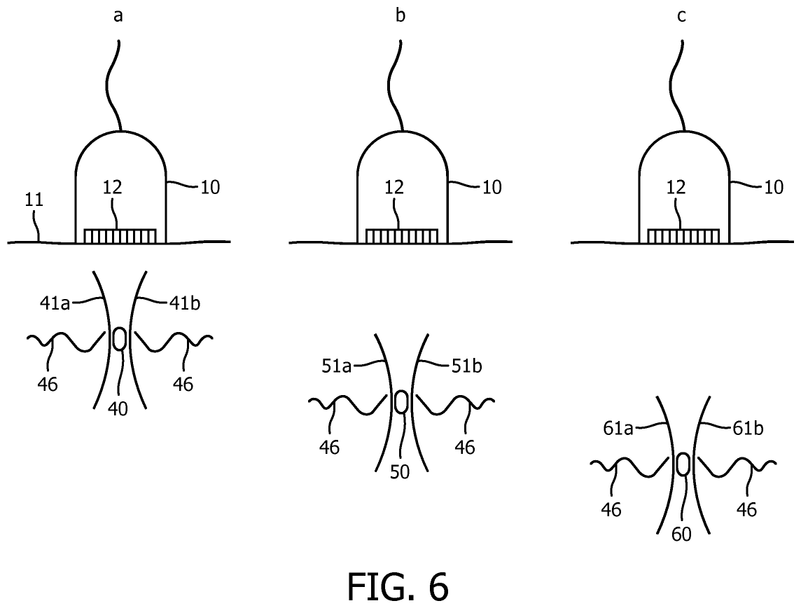

FIG. 6 illustrates the generation of shear waves by push pulses at three different depths in a region of interest.

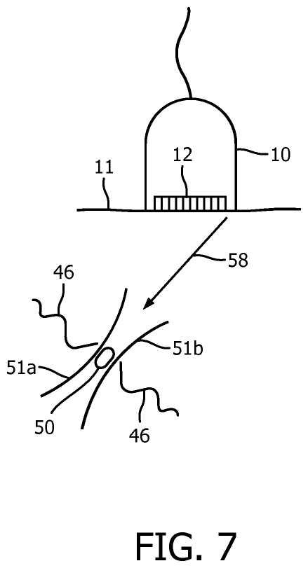

FIG. 7 illustrates the generation of shear waves by a push pulse angularly steered from a matrix array transducer probe.

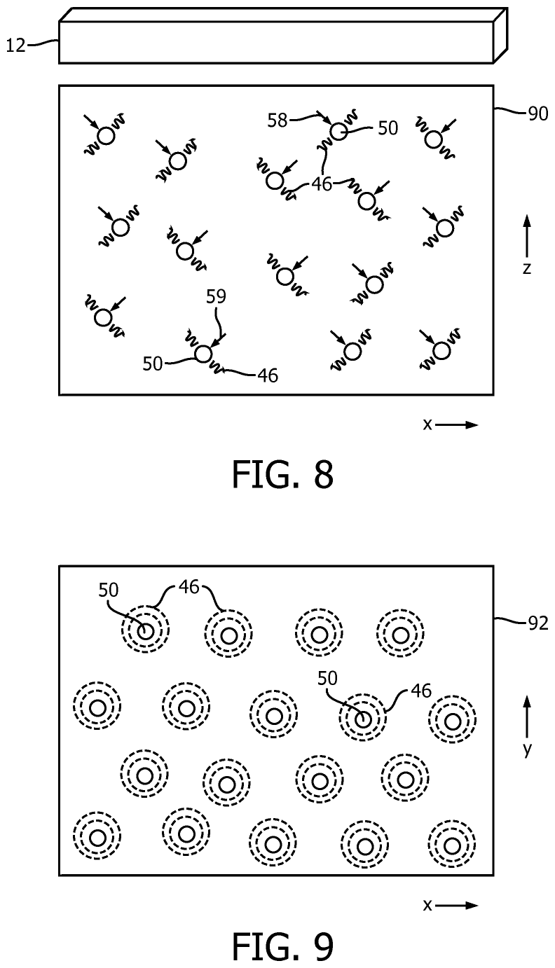

FIG. 8 illustrates the generation of shear waves in a B plane by transmission of differently steered push pulses.

FIG. 9 illustrates the generation of shear waves in a C plane below a matrix array transducer probe.

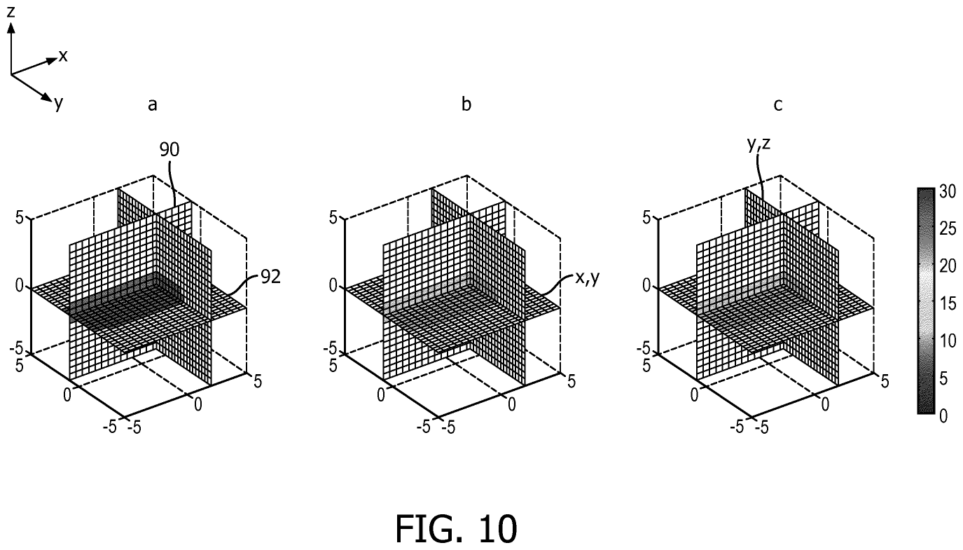

FIG. 10 illustrates three displays of planes of stiffness information through a region of interest in accordance with the principles of the present invention.

Referring first to FIG. 1, an ultrasound system constructed in accordance with the principles of the present invention for the measurement of stiffness using shear waves is shown in block diagram form. An ultrasound probe 10 has a two-dimensional matrix array 12 of transducer elements for transmitting and receiving ultrasound signals. A two-dimensional array transducer can scan a two-dimensional (2D) plane by transmitting beams and receiving returning echo signals over a single plane in the body and can also be used to scan a volumetric region by transmitting and receiving beams in different directions and/or planes of a volumetric (3D) region of the body. The array elements are coupled to a micro-beamformer 38 located in the probe which controls transmission by the elements and processes the echo signals received from groups or sub-arrays of elements into partially beamformed signals. The partially beamformed signals are coupled from the probe to a multiline receive beamformer 20 in the ultrasound system by a transmit/receive (T/R) switch 14. Coordination of transmission and reception by the beamformers is controlled by a beamformer controller 16 coupled to the multiline receive beamformer and to a transmit beamformer 18, which provides control signals to the micro-beamformer. The beamformer controller is responsive to signals produced in response to user manipulation of controls of a user interface or control panel 37 to control the operation of the ultrasound system and its probe.

The multiline receive beamformer 20 produces multiple, spatially distinct receive lines (A-lines) of echo signals during a single transmit-receive interval. The echo signals are processed by filtering, noise reduction, and the like by a signal processor 22, then stored in an A-line memory 24. A shear wave processor comprised of the following components 26-30 then processes the A-line data to determine velocity and/or stiffness values. Temporally distinct A-line samples relating to the same spatial vector location are associated with each other in an ensemble of echoes relating to a common point in the image field. The r.f. echo signals of successive A-line sampling of the same spatial vector are cross-correlated by an A-line r.f. cross-correlator 26 to produce a sequence of samples of tissue displacement for each sampling point on the vector. Alternatively, the A-lines of a spatial vector can be vector Doppler processed to detect shear wave induced tissue motion along the vector, or other phase-sensitive techniques such as speckle tracking in the time domain can be employed. A wavefront peak detector 28 is responsive to detection of the shear wave displacement along the A-line vectors to detect the peak of the shear wave tissue displacement at each sampling point on the A-line. In a preferred embodiment this is done by curve-fitting, although cross-correlation and other interpolative techniques can also be employed if desired. The times at which the peak of the shear wave displacement occurs is noted in relation to the times of the same event at other A-line locations, all to a common time reference, and this information is coupled to a wavefront velocity detector 30 which differentially calculates the shear wave velocity from the peak displacement times on adjacent A-lines. This velocity information is coupled into a velocity display map 32 stored in a memory which indicates the velocity of the shear wave at spatially different points in a 2D or 3D image field. The velocity display map is coupled to an image processor 34 which processes the velocity map for display on an image display 36. The display map can comprise shear wave velocity values at points in a region of interest, which values can be converted to other units of stiffness or viscosity such as shear elasticity modulus or Young's modulus values.

FIG. 2 is an illustration of the use of four acoustic radiation force push pulses to create a composite shear wavefront. The four push pulses are transmitted along vectors 44, 54, 64 and 74 which are seen to be aligned along a single vectorial direction in FIG. 2. When the shallowest push pulse of vector 44 is transmitted first, followed by successively deeper push pulses 54, 64, and 74, the shear wavefronts of the respective push pulses will have propagated as indicated by waves 46, 56, 66, and 76 by a time shortly after the last push pulse (vector 74) has been transmitted. As the shear waves 46, 56, 66, and 76 travel outward from the push pulse vector, they are interrogated by tracking pulses 80 shown in spatial sequence along the top of the drawing. Tracking pulses can occur between as well as after push pulses.

The velocity of a laterally traveling shear wave is detected by sensing the tissue displacement caused by the shear wave as it proceeds through the tissue. This is done with time-interleaved tracking pulses transmitted adjacent to the push pulse vector as shown in FIG. 3. In this example the push pulse(s) 40 is transmitted along push pulse vector 44 to generate a shear wave which travels perpendicular to the push pulse vector. A-line vectors adjacent to the push pulse vector 40 are sampled by sampling tracking pulses T1, T2, T3, T4, and T5 transmitted along each adjacent sampling pulse vector in a time-interleaved sequence. For example, the first vector location A1 is sampled by a first pulse T1, then the second vector location A2 by the next pulse T2, then A3, A4, and A5. Then vector location A1 is sampled again, and the sequence repeats at a pulse repetition frequency (PRF). The interval between pulse transmissions is referred to as the pulse repetition interval (PRI). Since the sampling is time-interleaved, each of the five vector locations is sampled once in every five sampling pulses in this example. In this example every vector location is pulsed fifty-five times for a total tracking time of 27.5 msec. Each pulse results in echoes returning from along the vector which are sampled by a high-speed A/D converter in the receive beamformer. Thus, for every sampled point along each vector there is an ensemble of 55 samples, with each sample taken at one-fifth the pulse rate of the T1-T5 sampling pulse sequence. The typical ensemble length at each echo location on a sampling vector is 40-100 samples. The sampling rate will be chosen in consideration of the frequency content of the shear wave displacement being detected so as to satisfy the Nyquist criterion for sampling. Since the purpose of the sampling is to sense and track the displacement effect of the shear wave as it progresses through the tissue, the vector locations may be located closer together for slowly moving shear waves and further apart for more rapidly moving shear waves. Other sequences of time-interleaving the vector sampling may also be employed. For example, odd-numbered vectors could be sampled in sequence, followed by sampling of the even-numbered vectors. As another example, vector locations A1-A3 could be sampled in a time-interleaved manner, then vector locations A2-A4, then vector locations A3-A5 to track the shear wave displacement as it progresses. Other sequences may also be employed based upon the exigencies of the situation. The ensembles of time-interleaved samples at each point along each sampling vector are then processed to find the time of peak tissue displacement at each spatially adjacent vector location.

In accordance with a preferred implementation, multiline transmission and reception is employed so that a single tracking pulse can simultaneously sample a plurality of adjacent, tightly spaced, A-line locations. Referring to FIG. 4, one technique for multiline transmission and reception is shown. In FIG. 4 a single A-line tracking pulse with a beam profile 82a, 82b is transmitted as indicated by the wide arrow A #. The broad beam profile insonifies multiple receive line locations as shown in the drawing. Preferably the tracking pulse is a so-called “fat pulse” as described in U.S. Pat. No. 4,644,795 (Augustine), for example. In this example four receive line locations A1-1, A1-2, A1-3 and A1-4 are insonified. Echoes from the four receive lines (4× multiline) are received in response to the single transmit pulse and are appropriately delayed and summed to produce coherent echo signals along each of the receive line locations. Beamformers capable of producing such simultaneous multilines are described, for instance, in U.S. Pat. No. 5,318,033 (Savord), U.S. Pat. No. 5,345,426 (Lipschutz), U.S. Pat. No. 5,469,851 (Lipschutz), U.S. Pat. No. 6,695,783 (Henderson et al.) and U.S. Pat. No. 8,137,272 (Cooley et al.) These multiline beamformers are typically used to decrease the acquisition time and thereby increase the frame rate of live ultrasound images, which is particularly useful when imaging the beating heart and blood flow in real time echocardiography. They are also useful in 3D ultrasound imaging so that real time frame rates of display can be attained. See, in this regard, U.S. Pat. No. 6,494,838 (Cooley et al.) In an implementation of the present invention, the benefit of multiline acquisition is two-fold: it enables a closely-spaced sampling line density and rapid acquisition of a short duration shear wave which only travels a short distance through tissue before being dissipated by attenuation. While higher order multiline may be employed which acquires samples along a greater number of A-lines at the same time and thus a higher sampling rate, this will require a broader transmit beam (A #) to simultaneously insonify the greater number of receive lines. The broader transmit beam will consequently diminish the signal-to-noise performance of the higher order implementation.

FIG. 3 illustrates the use of 4× multiline reception for transmission and reception along each sampling vector A1-A5. A first tracking pulse T₁ is transmitted close to the push pulse vector 44, insonifying four receive line locations A1-1 to A1-4 and four multiline A-lines are received in response from the lateral region A1. When the four multilines are centered with respect to the transmitted tracking pulse, echoes from two A-lines are received on each side of the center of the tracking pulse beam center, shown by A1-1 and A1-2 to the left of center and A1-3 and A1-4 to the right of center. In a preferred embodiment the A-lines are spaced 0.5 mm apart from each other. Shear waves generally move at a speed of 1-10 meters per second, and consequently tracking pulses are repetitively transmitted down lines A1-A5 in a time-interleaved manner and A-line samples received from the A-line locations during the time intervals between push pulses (when there are such intervals), and for 20 msec after the last push pulse, after which the shear wave has propagated out of the one centimeter A1-A5 sampling window. Since shear waves can have frequency components in the range of about 100 Hz to about 1000 Hz, sampling theory dictates that each A-line should have a sampling rate of 2 kHz. This results in a set (ensemble) of fifty-five A-line samplings of each sampling point on each multiline A-line.

In the example of FIG. 3, five tracking pulses, T₁-T₅, are transmitted over successive sampling windows A1-A5 adjacent to the push pulse vector 44 to sample the shear wave displacement effect as the wave propagates. A typical sampling pulse is a short pulse, usually only one or two cycles, at a frequency suitable for penetrating the depth being studied, such as 7-8 MHz. Each tracking pulse in this example is offset by 2 mm from its adjacent neighbors, resulting in twenty A-lines spaced 0.5 mm apart with 4× multiline over a total distance of one centimeter. There are various ways the interrogate the sampling windows. One is to just sample region A1 until the shear wave is detected, then to begin sampling in region A2, then A3, and so on. Another is to time interleave the sampling in the regions as described above, sampling with tracking pulses T₁-T₅ in succession, then repeating the sequence. With the latter approach five sampling windows with twenty tracking A-line positions can track the shear wave effect simultaneously. After the shear wave has passed through the closest A1 sampling window and into the adjacent windows, sampling of the near window can be terminated and the sampling time can be devoted to the remaining sampling windows through which the shear wave is still propagating. Sampling continues until the shear wave has propagated out of the one cm. sampling region, by which time the shear wave may have attenuated below a detectable level. Shear waves on average have a relaxation time of 10 msec.

It is necessary that the sampling times of the tracking A-line positions be related to a common time base when the tracking pulses are time-interleaved so that the results can be used to make a continuous measurement of time, and hence velocity, across the one cm. sampling region. For example, since the sampling pulses for sampling window A2 do not occur until 50 microseconds following the corresponding sampling pulses for window A1, a 50 microsecond time offset exists between the sampling times of the two adjacent windows. This time difference must be taken into account when comparing the peak times of displacement in the respective windows, and must be accounted for in an accumulated manner across the full one centimeter sampling window. Referencing the sampling times of each sampling vector to a common time reference can resolve the problem of the offset sampling times.

Since a diagnostic region-of-interest (ROI) is generally greater than one centimeter in width, the procedure of FIGS. 2-5 is repeated with push pulses transmitted at different spatial locations across the image field. An image field is thereby interrogated in one-centimeter wide regions, and the results of the interrogations are displayed adjacent to each other in anatomical relationship to present a stiffness image of the full ROI. A four-centimeter-wide image field can be interrogated in four adjacent or overlapping one cm. regions, for example, which are then displayed side-by-side or wholly or partially overlaid on the display.

FIG. 5 illustrates a sequence of displacement values for two laterally adjacent points of tissue on two adjacent A-lines such as A1-3 and A1-4 in FIG. 3. Curve 100 represents the displacement over time caused by passage of a shear wave through a point on A-line A1-3, and curve 120 the displacement at a point of adjacent A-line A1-4. Points 102-118 of tissue displacement values are calculated from local cross correlations of r.f. data (e.g., 10-30 r.f. samples in depth) acquired around a sampling point depth on A1-3 over time to yield the local displacement values over time at the depth point. The points 102-118 of displacement values detected at successive times (y-axis), when plotted as a function of time, are joined to form the first displacement curve 100. At a point on second A-line A-1-4 spaced to the right of the first A-line, the succession 122-136 of displacement values produced by local cross correlation can be joined to form a second displacement curve 120. Since the shear wave is traveling from left to right in this example, the second curve 120 for the right-most A-line is shifted to the right (in time, Δt) of the first displacement curve 100. A precise time reference of the passage of the wavefront from one point to the next is measured by the detected peak or inflection point of each displacement curve, indicated at 200 and 220 in this example. Various techniques can be used to find the curve peak. In one implementation the displacement values of each curve are processed by fitting curves to the values to form complete displacement curves 100, 120 and the curve peaks. Another technique is to interpolate additional points between the detected points to find the peak. Yet another technique is to determine the slopes of the curve on either side of the peak and determine the peak from the intersection of slope lines. Still another approach is cross-correlation of the curve data. When the peaks of the shear wave displacement at successive A-line positions are found by the waveform peak detector 28, their times of occurrence in relation to the detection of the points on the curves are noted. The difference of these times, Δt, taking into consideration sampling time offsets, and the spacing between the A-lines (e.g., 0.5 mm) is then used by the wavefront velocity detector 30 to determine the velocity of the shear wave as it traveled between the two A-line locations. After the entire ROI has been interrogated in this manner and displacement curves and times of peak occurrence determined for each sample point on each A-line vector, the velocity of shear wave travel can be calculated from one image point to another across the entire region of interest. This two- or three-dimensional matrix of velocity values is color-coded or otherwise coded with corresponding stiffness estimates to form a velocity or stiffness display map which is overlaid and in spatial alignment with a B mode image of the region of interest for display on image display 36.

In accordance with the principles of the present invention, the 2D matrix array transducer acquires stiffness data not from an entire 3D volume, but from three intersecting planes of the volume. How this is done for a B plane, a plane intersecting the face of the matrix array, is illustrated in the following drawings. FIG. 6 illustrates the transmission by a matrix array probe 10 of three push pulses, 40, 50, and 60, each at a different depth in an image field below the skinline 11. The beam profile of each push pulse is shown as 41a, 41b; 51a, 51b; and 61a, 61b. Each successive push pulse generates a shear wave 46 at a successively greater image depth. Three such push pulses can be transmitted in axial alignment to generate shear waves at three successive image depths. When the vectors of the push pulses are axially aligned, the result is that shear wave velocity is measured at three different depths in the z dimension along the same vector in the x (azimuth) dimension. When similar push pulses are transmitted and shear waves measured along a succession of depth directions in different azimuth directions x, shear wave velocity measurements are made in an x,z-oriented B plane.

In the example of FIG. 6, the generated shear waves all travel horizontally, since the push pulse vectors are oriented normal to the face of the matrix array and, as mentioned above, shear waves 46 always travel perpendicular to the axis of the vectors of push pulses 40, 50, and 60. Thus, the measurements of shear wave velocity are oriented horizontally and vertically across the sampled image plane. But the shear waves can be differently oriented by changing the orientation of the push pulse vectors as shown in FIG. 7. In this illustration the push pulse vector is oriented to the left as shown by arrow 58, which is done by steering the push pulse(s) to the left as shown in FIG. 7. With a 2D matrix array, push pulses can be steered in any direction in azimuth or elevation. Thus, a plurality of push pulses can be steered to the left as shown in a plane extending normal to the sheet of the drawing to make shear wave velocity measurements in a B plane aligned with the arrow 58. Alternatively, push pulses can be generated parallel to the arrow 58 across a plane which is in the plane of the drawing. It follows that, by means of beam steering, shear wave velocity measurements can be made in any B plane extending from the face of the matrix array 12 in any direction. When a push pulse 50 is transmitted as shown in FIG. 7, the resulting shear waves 46 travel outward from the shear wave focal point at an angle as shown in the drawing. In this example, this permits shear wave velocity measurements to be made to the upper left and lower right of the push pulse focus, by transmitting sampling pulses at the same angle as the push pulse vector 58.

With these push pulse transmission techniques in mind, it is seen that B planes of any orientation relative to the face of a 2D matrix array can be sampled for shear wave velocity measurement. FIG. 8 illustrates a B plane 90 extending from a matrix array 12 in an x,z plane. Shear wave velocities could be measured in this plane by transmitting push pulses vertically in the z direction, or in the z direction but steered in elevation, as illustrated in FIG. 6, but in this example shear waves are transmitted at two different angles 58 and 59 in the plane 90 as indicated by the small arrows in the drawing. This results in the generation of shear waves 46 which travel to the upper right and lower left, or to the lower left and upper right, as shown in the drawing. By transmitting a number of push pulses at push pulse focal points 50, shear waves are generated throughout the plane 90, which can be sampled by tracking pulses transmitted on either side of the push pulse focal points and their velocities determined throughout the plane 90. The beams for the push pulses and the tracking pulses can be steered in azimuth or elevation, so that the plane 90 can be oriented normal to or at any other angle relative to the face of the matrix array 12.

FIG. 9 illustrates a C plane 92 in the x,y plane in which shear wave velocities are measured in accordance with the present invention. A C plane is one which does not intersect the face of the matrix array 12. For instance, the C plane 92 could be parallel to the plane of the face of the matrix array as in this example. Shear waves are generated in the plane 92 by focusing push pulses at points 50 in the plane, causing shear waves to radiate outward as indicated by the dashed circles representing shear wavefronts. Tracking pulses can sample the radiating shear waves in any direction outward from a focal point, enabling shear wave velocities to be measured in multiple directions from a single push pulse. By targeting push pulses at intervals across the plane 92, shear wave velocities in the plane can be quickly and efficiently measured.

In accordance with the principles of the present invention, shear wave velocities are measured in three planes, which are displayed in an isometric or perspective view as illustrated in FIG. 10. In this example the three planes are all in an orthogonal relationship with each other, although planes oriented at nonorthogonal angles can also be used. Each of the examples shown in FIG. 10 has a vertical y,z plane, a vertical x,z plane, and a horizonal x,y plane, the directions taken with respect to the face of the matrix array transducer which scanned them. Shear wave velocity measurements are made in the vertical (B) planes using the acquisition techniques shown in FIG. 6 or 8, and velocity measurements are made in the horizontal (C) plane using the acquisition technique shown in FIG. 9, for instance. The velocity measurements are color-coded with stiffness values at their spatial pixel locations in the planes using a look-up table. The color bar to the right of FIG. 10 illustrates the range of stiffness-representing colors used in FIG. 10. In the example of FIG. 10a, higher stiffness is seen in the x-direction in C plane 92. The example of FIG. 10b illustrates greater stiffness values in the y-direction in the x,y plane, and the example of FIG. 10c illustrates greater stiffness values in the z-direction in the y,z plane.

In instances when the system is unable to acquire velocity data for some pixels in a plane, or a confidence map shows low confidence factors for regions of a plane, those ranges may be filled in with grayscale pixel data to illustrate the tissue structure when stiffness information is not displayed. See US pat. appl. No. [2017PF02765], Jago, for information on the use of confidence maps for shear wave imaging.

In accordance with a further aspect of the present invention, the relative planar locations are adjustable by a user. A user can click on a plane with the pointing device on the system user interface 37 and drag the plane to a different location. The user can click on the B plane 90, for instance, and drag it to the front or the back of the group of planes, or click on the C plane 92 and drag it to a higher or lower location in the display. The user can also click on a plane and tilt or rotate it relative to the other planes. When the spatial location of a plane is changed from the user interface, the change is registered with the beamformer controller 16, which then controls the beamformers 18, 20 and 38 and the matrix array 12 to acquire shear wave data from the new plane location. The graphics processor 42 also responds to the change by displaying the adjusted plane in its new spatial relation to the other displayed stiffness planes. Realtime frame rates of display can be achieved, because the data acquisition times needed are only those required to scan three planes of a volumetric region and not the entire 3D volume. The user can thereby examine an organ of the body like the liver by not only moving the probe to scan different regions of the liver, but can also reorient the planes which show stiffness values in the scan field of the matrix array. For instance, the user can view a B mode (tissue) image of the liver and spot a region of concern. The user can then position the shear wave scan planes with their common point of intersection in the center of the region. The three scan planes will thereby display stiffness variation in the region in three dimensions, left-to-right, top-to-bottom, and front-to-back. The user can also rotate the group of three planes to visualize all areas of the planes, in the same manner as a 3D dynamic parallax 3D display is rotated. The user can thus quickly and thoroughly assess the stiffness variation of an organ accurately and in real time.

Other variations will be evident to those skilled in the art. Rather than inducing the shear waves with acoustic radiation push pulses as described above, mechanical excitation from mechanical vibrators placed on the body around the probe can alternatively be used for shear wave generation. Another alternative is to use intrinsic physiological motion for shear wave generation, such as the pulse wave in the myocardium or blood vessels of the liver or other organs. Rather than acquiring the shear wave motion data with individual tracking pulses as described above, ultrafast 4-D acquisition can be performed to acquire an entire volume of r.f. data with each transmit event, then estimate shear wave displacement through volume-to-volume tracking of the 4-D datasets. Vector flow Doppler techniques can also be used to estimate shear wave displacement. The resultant data can be filtered with spatio-temporal filters or other filtering techniques to decompose the displacement/velocity signals into components along the directions of a plane. Physical or mechanical models can also be used to decompose displacement or velocity data into local physical parameters for display.

It should be noted that the ultrasound system of FIG. 1 which measures shear wave speed and derived measurements of stiffness may be implemented in hardware, software or a combination thereof. The various embodiments and/or components of an ultrasound system, for example, the modules, or components and controllers therein, also may be implemented as part of one or more computers or microprocessors. The computer or processor may include a computing device, an input device, a display unit and an interface, for example, for accessing the Internet. The computer or processor may include a microprocessor. The microprocessor may be connected to a communication bus, for example, to access a PACS system. The computer or processor may also include a memory. The memory devices such as the A-line memory 24 and the velocity map memory 32 may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” as used in describing components such as the signal processor 22, the image processor 34, and the graphics processor 42, may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine.

The set of instructions of an ultrasound system, including the shear wave generation, displacement measurement, and stiffness/velocity computations described above, may include various commands that instruct a computer or processor as a processing machine to perform specific operations such as the methods, computations, and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

1. An ultrasound imaging system for shear wave elastographic analysis comprising:
a two-dimensional matrix array transducer probe configured to receive echo signals from shear wave tissue displacement in a volumetric region of interest;
a shear wave processor, responsive to the echo signals from shear wave tissue displacement, and configured to produce measurements of tissue stiffness or velocity for points only in a selected set of three intersecting planes of the volumetric region of interest,
wherein the system comprises a microbeamformer, located in the transducer probe and coupled to the matrix array transducer, the microbeamformer configured to transmit push pulses to different points in said selected three intersecting planes; and
an image processor, coupled to the shear wave processor, and configured to display measurements of tissue stiffness or velocity in said three intersecting planes of the volumetric region of interest.

2. The ultrasound imaging system of claim 1, wherein the image processor is further configured to display color-coded measurements of tissue stiffness or velocity in the three intersecting planes of the volumetric region of interest.

3. The ultrasound imaging system of claim 2, wherein the image processor is further configured to display color-coded measurements of tissue stiffness or velocity in three orthogonally intersecting planes of the volumetric region of interest.

4. The ultrasound imaging system of claim 2, further comprising a user interface, coupled to the matrix array probe and the image processor, and adapted to control the relative orientation of the three planes.

5. The ultrasound imaging system of claim 2, wherein the three intersecting planes further comprise two B planes and one C plane.

6.-7. (canceled)

8. The ultrasound imaging system of claim 1, wherein the microbeamformer is further configured to steer transmitted push pulses in azimuth and elevation directions.

9. The ultrasound imaging system of claim 8, wherein the microbeamformer is further configured to transmit tracking pulses adjacent to push pulse focal points for shear wave displacement detection.

10. The ultrasound imaging system of claim 9, wherein the microbeamformer is further configured to receive echoes of shear wave tissue displacement in response to transmitted tracking pulses.

11. The ultrasound imaging system of claim 10, wherein the shear wave processor is further configured to process echoes of tissue displacement and produce measurements of shear wave velocity.

12. The ultrasound imaging system of claim 1, wherein the microbeamformer is configured to acquire a volume of r.f. data from the region of interest with each transmit event; and
wherein the shear wave processor is further configured to process volumes r.f. data from the region of interest to estimate shear wave displacement by volume-to-volume tracking of the r.f. datasets.

13. The ultrasound imaging system of claim 12, wherein the shear wave processor is further configured to filter the r.f. datasets with a spatiotemporal filter to decompose displacement signals into components along the directions of a plane.

14. The ultrasound imaging system of claim 13, further comprising a mechanical vibrator adapted to generate shear waves when located on a body adjacent to the matrix array transducer probe.

15. The ultrasound imaging system of claim 13, wherein the matrix array transducer probe is further configured to receive echo signals from shear wave tissue displacement caused by intrinsic physiological motion.
